SEQUENCE LISTING (4 sequences)

SEQ ID NO: 1 - DNA, 5784 bp, Homo sapiens
acggcatccagtacagaggggctggacttggacccctgcagcagccctgcacaggagaagcggcatataaagccgcgctgcccgggagccgctcggccacgtccaccggagcatcctgcactgcagggccggtctctcgctccagcagagcctgcgcctttctgactcggtccggaacactgaaaccagtcatcactgcatctttttggcaaaccaggagctcagctgcaggaggcaggatggtctggaggctggtcctgctggctctgtgggtgtggcccagcacgcaagctggtcaccaggacaaagacacgaccttcgaccttttcagtatcagcaacatcaaccgcaagaccattggcgccaagcagttccgcgggcccgaccccggcgtgccggcttaccgcttcgtgcgctttgactacatcccaccggtgaacgcagatgacctcagcaagatcaccaagatcatgcggcagaaggagggcttcttcctcacggcccagctcaagcaggacggcaagtccaggggcacgctgttggctctggagggccccggtctctcccagaggcagttcgagatcgtctccaacggccccgcggacacgctggatctcacctactggattgacggcacccggcatgtggtctccctggaggacgtcggcctggctgactcgcagtggaagaacgtcaccgtgcaggtggctggcgagacctacagcttgcacgtgggctgcgacctcataggaccagttgctctggacgagcccttctacgagcacctgcaggcggaaaagagccggatgtacgtggccaaaggctctgccagagagagtcacttcaggggtttgcttcagaacgtccacctagtgtttgaaaactctgtggaagatattctaagcaagaagggttgccagcaaggccagggagctgagatcaacgccatcagtgagaacacagagacgctgcgcctgggtccgcatgtcaccaccgagtacgtgggccccagctcggagaggaggcccgaggtgtgcgaacgctcgtgcgaggagctgggaaacatggtccaggagctctcggggctccacgtcctcgtgaaccagctcagcgagaacctcaagagagtgtcgaatgataaccagtttctctgggagctcattggtggccctcctaagacaaggaacatgtcagcttgctggcaggatggccggttctttgcggaaaatgaaacgtgggtggtggacagctgcaccacgtgtacctgcaagaaatttaaaaccatttgccaccaaatcacctgcccgcctgcaacctgcgccagtccatcctttgtggaaggcgaatgctgcccttcctgcctccactcggtggacggtgaggagggctggtctccgtgggcagagtggacccagtgctccgtgacgtgtggctctgggacccagcagagaggccggtcctgtgacgtcaccagcaacacctgcttggggccctcgatccagacacgggcttgcagtctgagcaagtgtgacacccgcatccggcaggacggcggctggagccactggtcaccttggtcttcatgctctgtgacctgtggagttggcaatatcacacgcatccgtctctgcaactccccagtgccccagatggggggcaagaattgcaaagggagtggccgggagaccaaagcctgccagggcgccccatgcccaatcgatggccgctggagcccctggtccccgtggtcggcctgcactgtcacctgtgccggtgggatccgggagcgcacccgggtctgcaacagccctgagcctcagtacggagggaaggcctgcgtgggggatgtgcaggagcgtcagatgtgcaacaagaggagctgccccgtggatggctgtttatccaacccctgcttcccgggagcccagtgcagcagcttccccgatgggtcctggtcatgcggcttctgccctgtgggcttcttgggcaatggcacccactgtgaggacctggacgagtgtgccctggtccccgacatctgcttctccaccagcaaggtgcctcgctgtgtcaacactcagcctggcttccactgcctgccctgcccgccccgatacagagggaaccagcccgtcggggtcggcctggaagcagccaagacggaaaagcaagtgtgtgagcccgaaaacccatgcaaggacaagacacacaactgccacaagcacgcggagtgcatctacctgggtcacttcagcgaccccatgtacaagtgcgagtgccagacaggctacgcgggcgacgggctcatctgcggggaggactcggacctggacggctggcccaacctcaatctggtctgcgccaccaacgccacctaccactgcatcaaggataactgcccccatctgccaaattctgggcaggaagactttgacaaggacgggattggcgatgcctgtgatgatgacgatgacaatgacggtgtgaccgatgagaaggacaactgccagctcctcttcaatccccgccaggctgactatgacaaggatgaggttggggaccgctgtgacaactgcccttacgtgcacaaccctgcccagatcgacacagacaacaatggagagggtgacgcctgctccgtggacattgatggggacgatgtcttcaatgaacgagacaattgtccctacgtctacaacactgaccagagggacacggatggtgacggtgtgggggatcactgtgacaactgccccctggtgcacaaccctgaccagaccgacgtggacaatgaccttgttggggaccagtgtgacaacaacgaggacatagatgacgacggccaccagaacaaccaggacaactgcccctacatctccaacgccaaccaggctgaccatgacagagacggccagggcgacgcctgtgaccctgatgatgacaacgatggcgtccccgatgacagggacaactgccggcttgtgttcaacccagaccaggaggacttggacggtgatggacggggtgatatttgtaaagatgattttgacaatgacaacatcccagatattgatgatgtgtgtcctgaaaacaatgccatcagtgagacagacttcaggaacttccagatggtccccttggatcccaaagggaccacccaaattgatcccaactgggtcattcgccatcaaggcaaggagctggttcagacagccaactcggaccccggcatcgctgtaggttttgacgagtttgggtctgtggacttcagtggcacattctacgtaaacactgaccgggacgacgactatgctggcttcgtctttggttaccagtcaagcagccgcttctatgtggtgatgtggaagcaggtgacgcagacctactgggaggaccagcccacgcgggcctatggctactccggcgtgtccctcaaggtggtgaactccaccacggggacgggcgagcacctgaggaacgcgctgtggcacacggggaacacgccggggcaggtgcgaaccttatggcacgaccccaggaacattggctggaaggactacacggcctataggtggcacctgactcacaggcccaagaccggctacatcagagtcttagtgcatgaaggaaaacaggtcatggcagactcaggacctatctatgaccaaacctacgctggcgggcggctgggtctatttgtcttctctcaagaaatggtctatttctcagacctcaagtacgaatgcagagatatttaaacaagatttgctgcatttccggcaatgccctgtgcatgccatggtccctagacacctcagttcattgtggtccttgcggcttctctctctagcagcacctcctgtcccttgaccttaactctgatggttcttcacctcctgccagcaaccccaaacccaagtgccttcagaggataaatatcaatggaactcagagatgaacatctaacccactagaggaaaccagtttggtgatatatgagactttatgtggagtgaaaattgggcatgccattacattgctttttcttgtttgtttaaaaagaatgacgtttacatataaaatgtaattacttattgtatttatgtgtatatggagttgaagggaatactgtgcataagccattatgataaattaagcatgaaaaatattgctgaactacttttggtgcttaaagttgtcactattcttgaattagagttgctctacaatgacacacaaatcccgctaaataaattataaacaagggtcaattcaaatttgaagtaatgttttagtaaggagagattagaagacaacaggcatagcaaatgacataagctaccgattaactaatcggaacatgtaaaacagttacaaaaataaacgaactctcctcttgtcctacaatgaaagccctcatgtgcagtagagatgcagtttcatcaaagaacaaacatccttgcaaatgggtgtgacgcggttccagatgtggatttggcaaaacctcatttaagtaaaaggttagcagagcaaagtgcggtgctttagctgctgcttgtgccgttgtggcgtcggggaggctcctgcctgagcttccttccccagctttgctgcctgagaggaaccagagcagacgcacaggccggaaaaggcgcatctaacgcgtatctaggctttggtaactgcggacaagttgcttttacctgatttgatgatacatttcattaaggttccagttataaatattttgttaatatttattaagtgactatagaatgcaactccatttaccagtaacttattttaaatatgcctagtaacacatatgtagtataatttctagaaacaaacatctaataagtatataatcctgtgaaaatatgaggcttgataatattaggttgtcacgatgaagcatgctagaagctgtaacagaatacatagagaataatgaggagtttatgatggaaccttaatatataatgttgccagcgattttagttcaatatttgttactgttatctatctgctgtatatggaattcttttaattcaaacgctgaaaacgaatcagcatttagtcttgccaggcacacccaataatcagtcatgtgtaatatgcacaagtttgtttttgtttttgttttttttgttggttggtttttttgctttaagttgcatgatctttctgcaggaaatagtcactcatcccactccacataaggggtttagtaagagaagtctgtctgtctgatgatggatagggggcaaatctttttcccctttctgttaatagtcatcacatttctatgccaaacaggaacgatccataactttagtcttaatgtacacattgcattttgataaaattaattttgttgtttcctttgaggttgatcgttgtgttgttttgctgcactttttacttttttgcgtgtggagctgtattcccgagacaacgaagcgttgggatacttcattaaatgtagcgactgtcaacagcgtgcaggttttctgtttctgtgttgtggggtcaaccgtacaatggtgtgggaatgacgatgatgtgaatatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacctcatttttaaagattaacaacaggaaataaattgtaaaaaaggttttct

SEQ ID NO: 2 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIGPVALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGFCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 3 - DNA, 17 bp, Homo sapiens
aatggaackcagagatg

SEQ ID NO: 4 - DNA, 17 bp, Homo sapiens
aaatgtagygactgtca

(19) United States
(12) Patent Application Publication
(10) Pub. No.: US 2005/0100953 A1
(43) Pub. Date: May 12, 2005

(54) Association of thrombospondin polymorphisms with vascular disease

(75) Inventors: Stacey Bolk, Lexington, MA (US); George Q. Daley, Weston, MA (US); Jeanette J. McCarthy, San Diego, CA (US)

Correspondence Address: FISH & NEAVE IP GROUP;ROPES & GRAY LLP, ONE INTERNATIONAL PLACE, BOSTON, MA, 02110-2624, US

(73) Assignees: Whitehead Institute for Biomedical Research; Millennium Pharmaceuticals, Inc.

(21) Appl. No.: 11/019,864

(22) Filed: Dec. 22, 2004

Related U.S. Application Data
(62) Division of application No. 10/007,781, filed on Nov. 13, 2001.
(60) Provisional application No. 60/248,130, filed on Nov. 13, 2000.
(60) Provisional application No. 60/300,158, filed on Jun. 22, 2001.

(51) Int. Cl. C12Q 1/68

(52) U.S. Cl. 435/6

(57) ABSTRACT

A role for the thrombospondin gene(s), particularly TSP-2, in vascular disease is disclosed. Use of single nucleotide polymorphisms in the thrombospondin gene(s) for diagnosis, prediction of clinical course and treatment response, development of therapeutics and development of cell-culture-based and animal models for research and treatment are disclosed.

### CROSS-REFERENCE TO RELATED APPLICATIONS

This application is a divisional of U.S. application Ser. No. 10/007,781, filed Nov. 13, 2001, which claims the benefit of U.S. Provisional Application No. 60/248,130, filed on Nov. 13, 2000 and U.S. Provisional Application No. 60/300,158, filed on Jun. 22, 2001. The entire teachings of the above applications are incorporated herein by reference.

### BACKGROUND OF THE INVENTION

The thrombospondins are a family of extracellular matrix (ECM) glycoproteins that modulate many cell behaviors including adhesion, migration, and proliferation. Thrombospondins (also known as thrombin sensitive proteins or TSPs) are large molecular weight glycoproteins composed of three identical disulfide-linked polypeptide chains. TSPs are stored in the alpha-granules of platelets and secreted by a variety of mesenchymal and epithelial cells (Majack et al., Cell Membrane 3:57-77 (1987)). Platelets secrete TSPs when activated in the blood by such physiological agonists such as thrombin. TSPs have lectin properties and a broad function in the regulation of fibrinolysis and as a component of the ECM, and are one of a group of ECM proteins which have adhesive properties. TSPs bind to fibronectin and fibrinogen (Lahav et al., Eur. J. Biochem. 145:151-6 (1984)), and these proteins are known to be involved in platelet adhesion to substratum and platelet aggregation (Leung, J Clin Invest 74:1764-1772 (1986)).

Recent work has implicated TSPs in response of cells to growth factors. Submitogenic doses of PDGF induce a rapid but transitory increase in TSP synthesis and secretion by rat aortic smooth muscle cells (Majack et al., J. Biol. Chem., 101: 1059-70 (1985)). PDGF responsiveness to TSP synthesis in glial cells has also been shown (Asch et al., Proc. Natl. Acad. Sci., 83:2904-8 (1986)). TSP mRNA levels rise rapidly in response to PDGF (Majack et al., J. Biol. Chem., 262:8821-5 (1987)). TSPs act synergistically with epidermal growth factor to increase DNA synthesis in smooth muscle cells (Majack et al., Proc. Natl. Acad. Sci., 83:9050-4 (1986)), and monoclonal antibodies to TSPs inhibit smooth muscle cell proliferation (Majack et al., J. Biol. Chem., 106:415-22 (1988)). TSPs modulate local adhesions in endothelial cells, and TSPs, particularly TSP-1 primarily derived from platelet granules, are known to be an important activator of transforming growth factor beta-1 (TGFB-1) (Crawford et al., Cell, 93:1159 (1998)) and appear to be a potential link between platelet-thrombosis and development of atherosclerosis.

### BRIEF SUMMARY OF THE INVENTION

The results described herein reveal an association between single nucleotide polymorphisms (SNPs) in TSP genes, particularly TSP-2, and vascular disease. In particular, SNPs in these genes which are associated with premature coronary artery disease (CAD)(or coronary heart disease) and myocardial infarction (MI) have been identified and represent a potentially vital marker of upstream biology influencing the complex process of atherosclerotic plaque generation and vulnerability.

Thus, the invention relates to the SNPs identified as described herein, both singly and in combination, as well as to the use of these SNPs, and others in TSP genes, particularly those nearby in linkage disequilibrium with these SNPs, for diagnosis, prediction of clinical course and treatment response for vascular disease, development of new treatments for vascular disease based upon comparison of the variant and normal versions of the gene or gene product, and development of cell-culture based and animal models for research and treatment of vascular disease. The invention further relates to novel compounds and pharmaceutical compositions for use in the diagnosis and treatment of such disorders. In preferred embodiments, the vascular disease is CAD or MI.

The invention relates to isolated nucleic acid molecules comprising all or a portion of the variant allele of TSP-2 (e.g., as exemplified by SEQ ID NO: 1). Preferred portions are at least 10 contiguous nucleotides and comprise the polymorphic site, e.g., a portion of SEQ ID NO: 1 which is at least 10 contiguous nucleotides and comprises the “G” at position 3949. The invention further relates to isolated gene products, e.g., polypeptides or proteins, which are encoded by a nucleic acid molecule comprising all or a portion of the variant allele of TSP-2 (e.g., SEQ ID NO: 1).

The invention further relates to a method of diagnosing or aiding in the diagnosis (or predicting the likelihood) of a disorder associated with the presence of a T at nucleotide position 3949 of SEQ ID NO: 1 in an individual. The method comprises obtaining a nucleic acid sample from the individual and determining the nucleotide present at nucleotide position 3949. The nucleic acid sample from the individual is assessed to determine whether the individual is homozygous (for either the alternate or reference form) or heterozygous. An individual who is heterozygous (i.e., having one copy of each allele, e.g., GT) at nucleotide position 3949 has an increased likelihood of said disorder (or an increased likelihood of having severe symptomology) as compared with an individual who is homozygous for the reference allele (TT). An individual who is homozygous for the variant allele (GG) has a decreased likelihood of said disorder (or a decreased likelihood of having severe symptomology) as compared with an individual who is homozygous for the reference allele (TT). In a particular embodiment the disorder is a vascular disease selected from the group consisting of atherosclerosis, coronary heart disease, myocardial infarction (MI), stroke, peripheral vascular diseases, venous thromboembolism and pulmonary embolism. In a preferred embodiment, the vascular disease is selected from the group consisting of CAD and MI. In a particular embodiment, the individual is an individual at risk for development of a vascular disease.

In another embodiment, the invention relates to pharmaceutical compositions comprising a variant TSP-2 gene or gene product, or active portion thereof, for use in the treatment of vascular diseases. The invention further relates to the use of agonists and antagonists of TSP-2 activity for use in the treatment of vascular diseases. In a particular embodiment the vascular disease is selected from the group consisting of atherosclerosis, coronary heart disease, myocardial infarction (MI), stroke, peripheral vascular diseases, venous thromboembolism and pulmonary embolism. In a preferred embodiment, the vascular disease is selected from the group consisting of CAD and MI.

### BRIEF DESCRIPTION OF THE DRAWINGS

FIGS. 1a-1d show the reference nucleotide (SEQ ID NO: 1) and amino acid (SEQ ID NO: 2) sequences for TSP-2, along with additional information obtained from Genbank.

FIG. 2 shows the results of an analysis of the association between SNPs in the TSP-1, TSP-2 and TSP-4 genes and vascular disorders.

### DETAILED DESCRIPTION OF THE INVENTION

The thrombospondin family of five proteins are known to play a pivotal role in modulating vascular injury, interaction with matrix, modulating coagulation, matrix interactions, angiogenesis, and serving as a key ligand for CD36, the oxidized LDL receptor, and the αᵥβ₃ integrins (Simantov, R., et al., “Histidine-rich glycoprotein inhibits the antiangiogenic effect of thrombospondin-1,” The Journal of Clinical Investigation, 107:45-52 (2001); Lawler, J. and R. O. Hynes, “The structure of human thrombospondin, an adhesive glycoprotein with multiple calcium-binding sites and homologies with several different proteins,” Journal of Cell Biology, 103:1635-1648 (1986); O'Rourke, K. M., et al., “Thrombospondin 1 and thrombospondin 2 are expressed as both homo- and heterotrimers,” Journal of Biological Chemistry, 267:24921-24924 (1992); Laherty, C. D., et al., “Characterization of mouse thrombospondin 2 sequence and expression during cell growth and development,” Journal of Biological Chemistry, 267:3274-3281 (1992); Lawler, J., “Characterization of human thrombospondin-4,” The Journal of Biological Chemistry, 270:2809-2814 (1995); Bornstein, P., “Diversity of function is inherent in matricellular proteins: An appraisal of thrombospondin 1,” Journal of Cell Biology, 130:503-506 (1995); LaBell, T. L., et al., “Sequence and characterization of the complete human thrombospondin 2 cDNA: Potential regulatory role for the 3 untranslated region,” Genomics, 17:225-229 (1993)). Thrombospondin can be synthesized and secreted by platelets, and using immunohistochemistry, thrombospondin has been demonstrated in atherosclerotic plaque (Wight, T. N., et al., “Light microscopic immunolocation of thrombospondin in human tissues,” The Journal of Histochemistry and Cytochemistry, 33:295-302 (1985) and Riessen, R., et al., “Cartilage oligomeric matrix protein (thrombospondin-5) is expressed by human vascular smooth muscle cells,” Artheriosclerosis, Thrombosis and Vascular Biology, 21:47-54 (2001)). Recent experiments with mice in thrombospondin-2 have shown this protein to be critical in cell-matrix interactions, and specifically matrix metalloproteinase-2; a deficiency in this protein led to high levels of this enzyme implicated in the vulnerability of atherosclerotic plaque (Kyriakides, T. R., et al., “Mice that lack thrombospondin 2 display connective tissue abnormalties that are associated with disordered collagen fibrillogenesis, an increased vascular density, and a bleeding diathesis,” The Journal of Cell Biology, 140:419-430 (1998) and Yang, Z., et al., “Matricellular proteins as modulators of cell-matrix interactions: Adhesive defect in thrombospondin 2-null fibroblasts is a consequence of increased levels of matrix matalloproteinase-2,” Molecular Biology of the Cell, 11:3353-3364 (2000)). Mutations in the type 3repeats, such as those identified in thrombospondin-4, would be expected to affect folding and secretion of the protein that normally exists as a pentamer. Indeed, the predicted secondary protein structure of the thrombospondin-4 variant suggests a significant disruption of the calcium binding site (Lawler, J. and R. O. Hynes. ibid.; Bornstein, P. and E. H. Sage, “Thrombospondins,” Methods in Enzymology, 245:62-84 (1994); and Bornstein, P., “Thrombospondins: Structure and regulation of Expression,” FASEB Journal, 6:3290-3299 (1992)). A mutation of the type 3 unit of thrombospondin-5, also known as cartilage oligomeric matrix protein, has been shown to cause pseudochondroplasia and multiple epiphyseal dysplasia (Briggs, M. D., et al., Pseudoachondroplasia and multiple epiphyseal dysplasis due to mutations in the cartillage oligomeric matrix protein gene,” Nature Genetics, 10:330-336 (1995)). Zhao and colleagues have recently shown a marked association of allograft vasalopathy in heart transplant patients (Zhao, X-M., et al., “Associations of thrombospondin-I and cardiac allograft vasculopathy in human cardiac allografts,” Circulation, 103:525-531 (2001)). Indeed, it is clear that the thrombosis proteins, as a family, function in thrombosis, and may be particularly well suited to play a major role, if altered, in premature atherosclerosis and myocardial infarction (Zhao, X-M., et al., ibid. and Crawford, S. E., et al., “Thrombospondin-l is a major activator of TGF-β1 in vivo,” Cell, 93:1159-1170 (1998)).

Recent advances in high throughput genomics technology have enabled our ability to catalogue allelic variants in large sets of candidate genes related to disease pathophysiology, and to test their relevance in genetic association of studies of defined patient populations.

A total of 420 families consisting of 1366 patients with premature coronary artery disease were identified in 15 participating medical centers, fulfilling the criteria of either myocardial infarction, revascularization, or a significant coronary artery lesion diagnosed before age 45 in men or age 50 in women. The sibling with earliest onset in a Caucasian subset of these families was compared with a random sample of 418 Caucasian controls with known coronary disease. A total of 62 vascular biology genes and 85 single-nucleotide polymorphisms (SNPs) were assessed.

A variant in the 3′ untranslated region of thrombospondin-2 (change of thymidine to guanine) had a protective effect against MI in individuals homozygous for the variant (adjusted odds ratio of 0.27; p=0.0.011).

One of the most important risk factors for coronary artery disease (CAD) is a familial history. Although family history subsumes both genetic and shared environmental factors, a study of twins with CAD suggests that CAD has a very strong genetic component, especially in patients who develop the disease at young ages (Marenberg, New England Journal of Medicine (1994)). Premature CAD signifies a particular advanced, malignant form of artherosclerotic heart disease, manifest at least a decade before the typical age of 55 to 65 years for initial presentation. Despite the importance of family history as a risk factor for coronary heart disease, its complex basis has not been elucidated. Unlike other complex diseases, few family-based studies have been carried out to identify genomic regions linked to CAD. The only published results to date on a genomic-wide scan for premature CAD loci identified two candidate regions linked to, premature Xq23-26 (PAJUKANTA 200). The relevant genes in these intervals have not been identified.

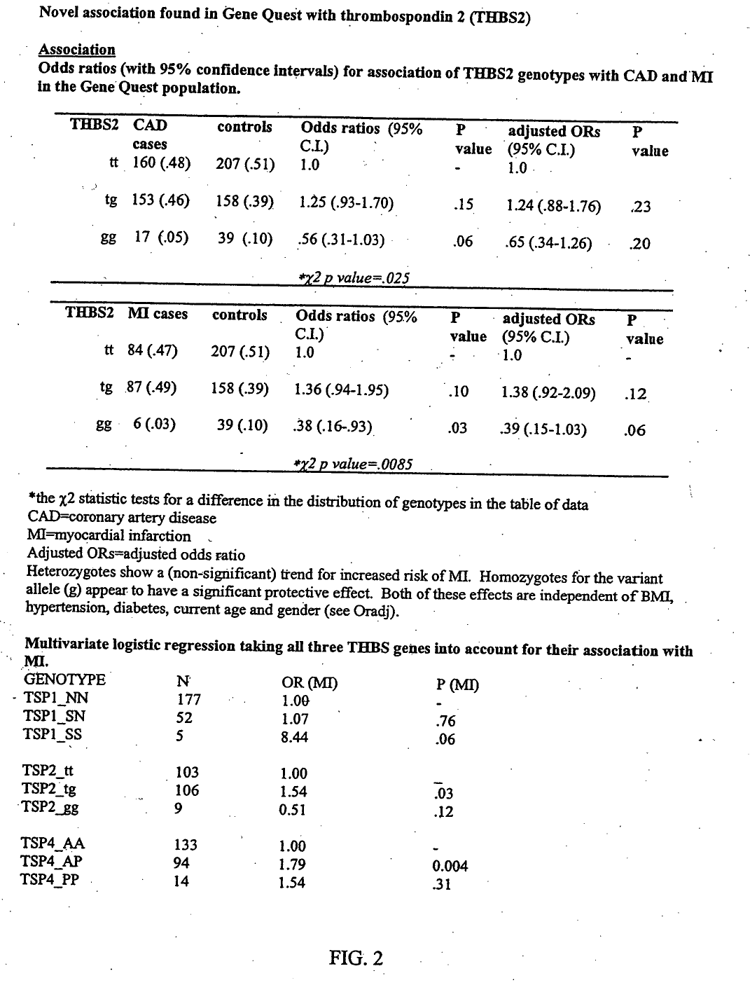

As described herein, a statistically significant association has been identified between a SNP (WFGC polyid G5755e5) in the thrombospondin-(TSP) 2 gene and vascular disorders (e.g., premature CAD and MI). In particular, a SNP (T to G) at nucleotide position 3949 in the TSP-2 gene (e.g., SEQ ID NO: 1) has been analyzed. The results of this analysis are shown in the upper portion of FIG. 2. The results show that an individual who is heterozygous (GT) at nucleotide position 3949 has an increased likelihood of said disorder (or an increased likelihood of having severe symptomology) as compared with an individual who is homozygous for the reference allele (TT). The results also show that an individual who is homozygous for the variant allele (GG) has a decreased likelihood of said disorder (or a decreased likelihood of having severe symptomology) as compared with an individual who is homozygous for the reference allele (TT). This SNP is located in the 3′ untranslated region, near a highly conserved region thought to have a potential regulatory role (LaBell et al., Genomics, 17:225-229 (1993)).

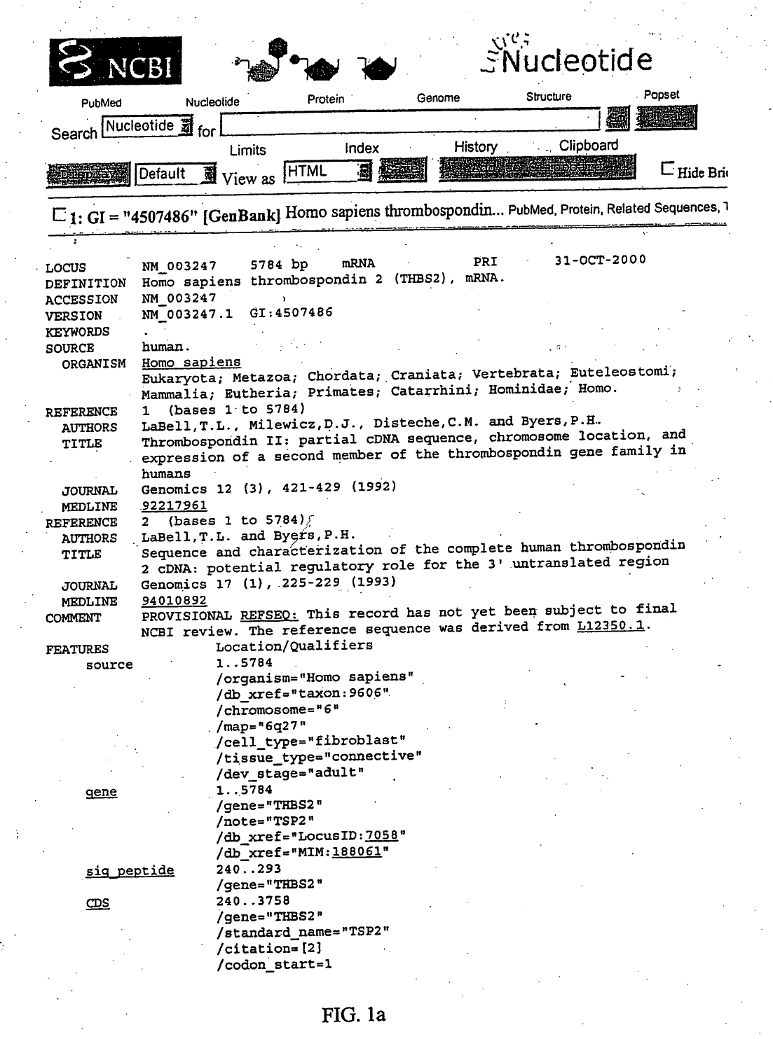

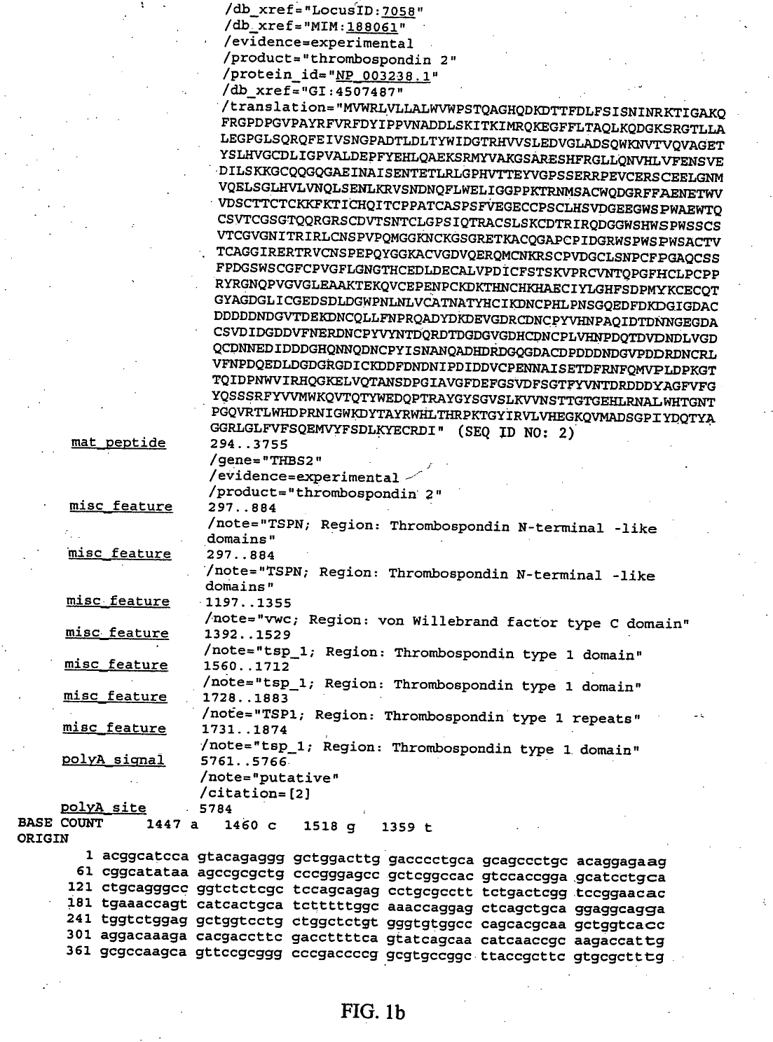

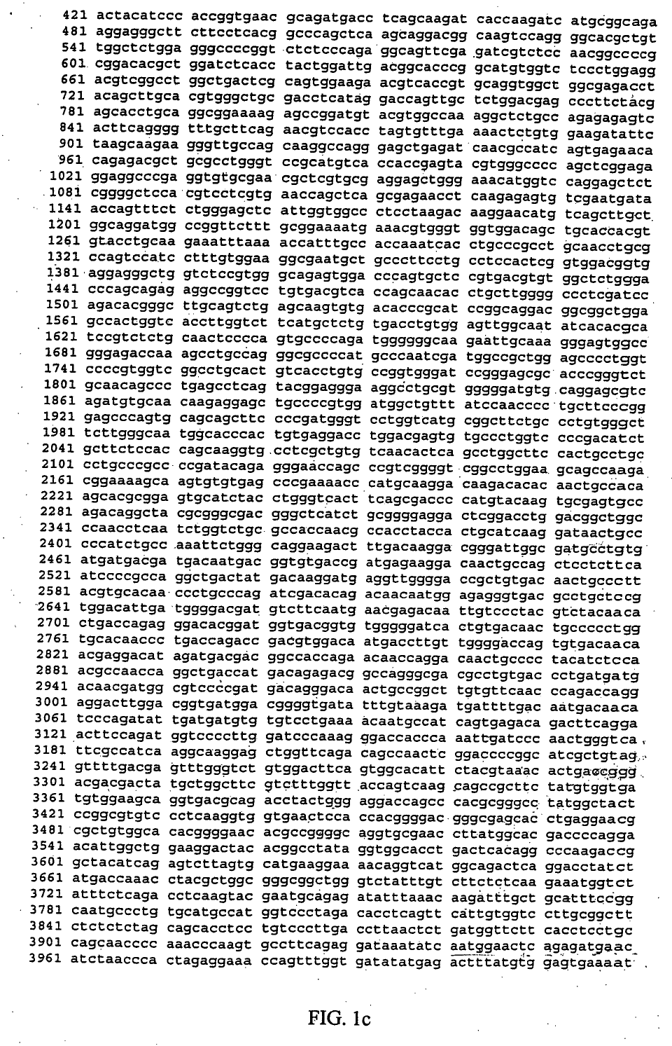

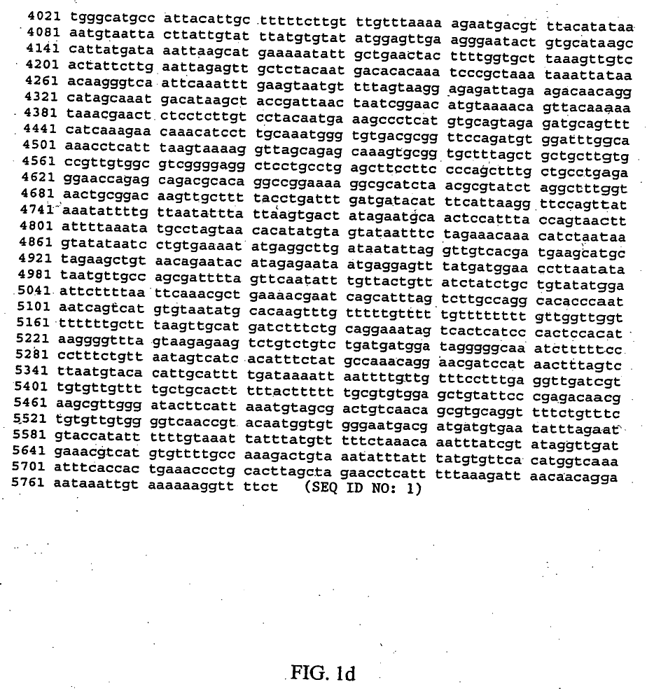

Specific reference nucleotide (SEQ ID NO: 1) and amino acid (SEQ ID NO: 2) sequences for TSP-2 as shown in Genbank are shown in FIGS. 1a-1d. It is understood that the invention is not limited by these exemplified reference sequences, as variants of these sequences which differ at locations other than the SNP sites identified herein can also be utilized. The skilled artisan can readily determine the SNP sites in these other reference sequences which correspond to the SNP sites identified herein by aligning the sequence of interest with the reference sequences specifically disclosed herein, and programs for performing such alignments are commercially available. For example, the ALIGN program in the GCG software package can be used, utilizing a PAM120 weight residue table, a gap length penalty of 12 and a gap penalty of 4, for example.

As used herein, the term “polymorphism” refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair, in which case it is referred to as a single nucleotide polymorphism (SNP).

Thus, the invention relates to a method for predicting the likelihood that an individual will have a vascular disease, or for aiding in the diagnosis of a vascular disease, or predicting the likelihood of having altered symptomology associated with a vascular disease, comprising the steps of obtaining a DNA sample from an individual to be assessed and determining the nucleotide present at nucleotide position 3949 of the TSP-2 gene. In one embodiment the TSP-2 gene has the nucleotide sequence of SEQ ID NO: 1. In a preferred embodiment of the invention, the individual is assessed to determine whether he or she is heterozygous or homozygous (reference or wildtype) at nucleotide position 3949. An individual who is heterozygous (GT) at nucleotide position 3949 has an increased likelihood of said disorder (or an increased likelihood of having severe symptomology) as compared with an individual who is homozygous for the reference allele (TT). An individual who is homozygous for the variant allele (GG) has a decreased likelihood of said disorder (or a decreased likelihood of having severe symptomology) as compared with an individual who is homozygous for the reference allele (TT).

In a particular embodiment, the individual is an individual at risk for development of a vascular disease. In another embodiment the individual exhibits clinical symptomology associated with a vascular disease. In one embodiment, the individual has been clinically diagnosed as having a vascular disease. Vascular diseases include, but are not limited to, atherosclerosis, coronary heart disease, myocardial infarction (MI), stroke, peripheral vascular diseases, venous thromboembolism and pulmonary embolism. In preferred embodiments, the vascular disease is CAD or MI.

The genetic material to be assessed can be obtained from any nucleated cell from the individual. For assay of genomic DNA, virtually any biological sample (other than pure red blood cells) is suitable. For example, convenient tissue samples include whole blood, semen, saliva, tears, urine, fecal material, sweat, skin and hair. For assay of cDNA or mRNA, the tissue sample must be obtained from a tissue or organ in which the target nucleic acid is expressed.

Many of the methods described herein require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The nucleotide which occupies the polymorphic site of interest (e.g., nucleotide position 3949 in TSP-2) can be identified by a variety of methods, such as Southern analysis of genomic DNA; direct mutation analysis by restriction enzyme digestion; Northern analysis of RNA; denaturing high pressure liquid chromatography (DHPLC); gene isolation and sequencing; hybridization of an allele-specific oligonucleotide with amplified gene products; single base extension (SBE). In a preferred embodiment, determination of the allelic form of TSP is carried out using SBE-FRET methods as described herein, or using chip-based oligonucleotide arrays as described herein. A sampling of suitable procedures is discussed below in turn.

1. Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2. Tiling Arrays

The polymorphisms can also be identified by hybridization to nucleic acid arrays, some examples of which are described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations-are expected to occur within a short distance commensurate with the length of the probes (e.g., two or more mutations within 9 to 21 bases).

3. Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17:2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

4. Direct-Sequencing

The direct analysis of the sequence of polymorphisms of the present invention can be accomplished using either the dideoxy chain termination method or the Maxam Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

6. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci., 86:2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

7. Single-Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997), incorporated herein by reference) uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

The polymorphisms of the invention may be associated with vascular disease in different ways. The polymorphisms may exert phenotypic effects indirectly via influence on replication, transcription, and translation. Additionally, the described polymorphisms may predispose an individual to a distinct mutation that is causally related to a certain phenotype, such as susceptibility or resistance to vascular disease and related disorders. The discovery of the polymorphisms and their correlation with CAD and MI facilitates biochemical analysis of the variant and reference forms of the gene and the development of assays to characterize the variant and reference forms and to screen for pharmaceutical agents that interact directly with one or another form.

Alternatively, these particular polymorphisms may belong to a group of two or more polymorphisms in the TSP gene(s) which contributes to the presence, absence or severity of vascular disease. An assessment of other polymorphisms within the TSP gene(s) can be undertaken, and the separate and combined effects of these polymorphisms, as well as alternations in other, distinct genes, on the vascular disease phenotype can be assessed. For example, SNPs in the TSP-1 and TSP-4 genes and their association with vascular disease are described in U.S. Provisional applications by Bolk et al., Ser. Nos. 60/220,947 and 60/225,724, filed Jul. 26, 2000 and Aug. 16, 2000, respectively, and in U.S. application Ser. No. 09/657,472, filed Sep. 7, 2000, by Lander et al. The teachings of these applications are incorporated herein by reference in their entirety. An analysis of the TSP-2 SNPs in combination with the TSP-1 and TSP-4 SNPs is shown in the lower portion of FIG. 2.

Correlation between a particular phenotype, e.g., the CAD or MI phenotype, and the presence or absence of a particular allele is performed for a population of individuals who have been tested for the presence or absence of the phenotype. Correlation can be performed by standard statistical methods such as a Chi-squared test and statistically significant correlations between polymorphic form(s) and phenotypic characteristics are noted. This correlation can be exploited in several ways. In the case of a strong correlation between a particular polymorphic form, e.g., the variant allele for TSP-2, and a disease for which treatment is available, detection of the polymorphic form in an individual may justify immediate administration of treatment, or at least the institution of regular monitoring of the individual. Detection of a polymorphic form correlated with a disorder in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic form and a particular disorder, immediate therapeutic intervention or monitoring may not be justified. Nevertheless, the individual can be motivated to begin simple life-style changes (e.g., diet modification, therapy or counseling) that can be accomplished at little cost to the individual but confer potential benefits in reducing the risk of conditions to which the individual may have increased susceptibility by virtue of the particular allele. Furthermore, identification of a polymorphic form correlated with enhanced receptiveness to one of several treatment regimes for a disorder indicates that this treatment regimen should be followed for the individual in question.

Furthermore, it may be possible to identify a physical linkage between a genetic locus associated with a trait of interest such as CAD or MI and polymorphic markers that are or are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA), 83:7353-7357 (1986); Lander et al., Proc. Natl. Acad. Sci. (USA), 84:2363-2367 (1987); Donis-Keller et al., Cell, 51:319-337 (1987); Lander et al., Genetics, 121:185-199 (1989)). Genes localized by linkage can be cloned by a process known as directional cloning. See Wainwright, Med. J. Australia, 159:170-174 (1993); Collins, Nature Genetics, 1:3-6 (1992).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science, 245:1073-1080 (1989); Monaco et al., Nature, 316:842 (1985); Yamoka et al., Neurology, 40:222-226 (1990); Rossiter et al., FASEB Journal, 5:21-27 (1991).

Linkage is analyzed by calculation of LOD (log of the odds) values. A LOD value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5th ed, W.B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is: probability of data if loci linked at θ to probability of data if loci unlinked. The computed likelihoods are usually expressed as the log₁₀ of this ratio (i.e., a LOD score). For example, a LOD score of 3 indicates 1000:1 odds against an apparent observed linkage being a coincidence. The use of logarithms allows data collected from different families to be combined by simple addition. Computer programs are available for the calculation of LOD scores for differing values of θ (e.g., LIPED, MLINK (Lathrop, Proc. Nat. Acad. Sci. (USA) 81:3443-3446 (1984)). For any particular LOD score, a recombination fraction may be determined from mathematical tables. See Smith et al., Mathematical tables for research workers in human genetics (Churchill, London, 1961); Smith, Ann. Hum. Genet. 32:127-150 (1968). The value of 0 at which the LOD score is the highest is considered to be the best estimate of the recombination fraction.

Positive LOD score values suggest that the two loci are linked, whereas negative values suggest that linkage is less likely (at that value of θ) than the possibility that the two loci are unlinked. By convention, a combined LOD score of +3 or greater (equivalent to greater than 1000:1 odds in favor of linkage) is considered definitive evidence that two loci are linked. Similarly, by convention, a negative LOD score of −2 or less is taken as definitive evidence against linkage of the two loci being compared. Negative linkage data are useful in excluding a chromosome or a segment thereof from consideration. The search focuses on the remaining non-excluded chromosomal locations.

In another embodiment, the invention relates to pharmaceutical compositions comprising a reference or variant TSP-2 gene or gene product for use in the treatment of vascular disease, such as CAD and MI. As used herein, a reference or variant TSP-2 gene product is intended to mean gene products which are encoded by the reference or variant allele, respectively, of the TSP-2 gene. In addition to substantially full-length polypeptides expressed by the genes, the present invention includes biologically active fragments of the polypeptides, or analogs thereof, including organic molecules which simulate the interactions of the peptides. Biologically active fragments include any portion of the fill-length polypeptide which confers a biological function on the variant gene product, including ligand binding, and antibody binding. Ligand binding includes binding by nucleic acids, proteins or polypeptides, small biologically active molecules, or large cellular structures.

For instance, the polypeptide or protein, or fragment thereof, of the present invention can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to procedures well known to medicinal chemists, and will depend on the ultimate pharmaceutical formulation desired. Methods of introduction of exogenous peptides at the site of treatment include, but are not limited to, intradermal, intramuscular, intraperitoneal, intravenous, subcutaneous, oral and intranasal. Other suitable methods of introduction can also include rechargeable or biodegradable devices and slow release polymeric devices. The pharmaceutical compositions of this invention can also be administered as part of a combinatorial therapy with other agents and treatment regimens.

The invention further pertains to compositions, e.g., vectors, comprising a nucleotide sequence encoding reference or variant TSP-2 gene products. For example, reference genes can be expressed in an expression vector in which a reference gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like.

It is also contemplated that cells can be engineered to express the reference allele of the invention by gene therapy methods. For example, DNA encoding the reference TSP gene product, or an active fragment or derivative thereof, can be introduced into an expression vector, such as a viral vector, and the vector can be introduced into appropriate cells in an animal. In such a method, the cell population can be engineered to inducibly or constitutively express active reference TSP gene product. In a preferred embodiment, the vector is delivered to the bone marrow, for example as described in Corey et al. (Science, 244:1275-1281 (1989)).

The invention further relates to the use of compositions (i.e., agonists) which enhance or increase the activity of the reference (or variant) TSP-2 gene product, or a functional portion thereof, for use in the treatment of vascular disease. The invention also relates to the use of compositions such as antagonists which reduce or decrease the activity of the variant (or reference) TSP-2 gene product, or a functional portion thereof, for use in the treatment of vascular disease.

The invention also relates to constructs which comprise a vector into which a sequence of the invention has been inserted in a sense or antisense orientation. For example, a vector comprising a nucleotide sequence which is antisense to the reference TSP-2 allele may be used as an antagonist of the activity of the TSP-2 reference allele. Alternatively, a vector comprising a nucleotide sequence of the TSP-2 variant allele may be used therapeutically to treat vascular diseases. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequencers) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and other factors.

The expression vectors of the invention can be introduced into host cells to thereby produce proteins or peptides, including fusion proteins or peptides, encoded by nucleic acids as described herein. The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a nucleic acid of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of the invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid of the invention has been introduced. Such host cells can then be used to create non-human transgenic animals in which exogenous nucleotide sequences have been introduced into their genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a nucleic acid of the invention into the male pronuclei of a fertilized oocyte, e.g., by microinjection, retroviral infection, and allowing the oocyte to develop in a pseudopregnant female foster animal. The sequence can be introduced as a transgene into the genome of a non-human animal. Intronic sequences and polyadenylation signals can also be included in the transgene to increase the efficiency of expression of the transgene. A tissue-specific regulatory sequence(s) can be operably linked to the transgene to direct expression of a polypeptide in particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA in tissues or cells of the animals. A transgenic founder animal can then be used to breed additional animals carrying the transgene. Moreover, transgenic animals carrying a transgene encoding the transgene can further be bred to other transgenic animals carrying other transgenes.

The invention also relates to the use of the variant and reference gene products to guide efforts to identify the causative mutation for vascular diseases or to identify or synthesize agents useful in the treatment of vascular diseases, e.g., CAD and MI. Amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro, or in vitro activity. Sites that are critical for polypeptide activity can also be determined by structural analysis such as crystallization, nuclear magnetic resonance or photoaffinity labeling (Smith et al., J. Mol. Biol., 224:899-904 (1992); de Vos et al., Science, 255:306-312 (1992)).

Another aspect of the invention pertains to monitoring the influence of agents (e.g., drugs, compounds) on the expression or activity of proteins of the invention in clinical trials. An exemplary method for detecting the presence or absence of proteins or nucleic acids of the invention in a biological sample involves obtaining a biological sample from a test subject and contacting the biological sample with a compound or an agent capable of detecting the protein, or nucleic acid (e.g., mRNA, genomic DNA) that encodes the protein, such that the presence of the protein or nucleic acid is detected in the biological sample. A preferred agent for detecting mRNA or genomic DNA is a labeled nucleic acid probe capable of hybridizing to mRNA or genomic DNA sequences described herein, preferably in an allele-specific manner. The nucleic acid probe can be, for example, a full-length nucleic acid, or a portion thereof, such as an oligonucleotide of at least 15, 30, 50, 100, 250 or 500 nucleotides in length and sufficient to specifically hybridize under stringent conditions to appropriate mRNA or genomic DNA. Other suitable probes for use in the diagnostic assays of the invention are described herein.

The invention also encompasses kits for detecting the presence of proteins or nucleic acid molecules of the invention in a biological sample. For example, the kit can comprise a labeled compound or agent (e.g., nucleic acid probe) capable of detecting protein or mRNA in a biological sample; means for determining the amount of protein or mRNA in the sample; and means for comparing the amount of protein or mRNA in the sample with a standard. The compound or agent can be packaged in a suitable container. The kit can further comprise instructions for using the kit to detect protein or nucleic acid.

Exemplification

A case-control study was undertaken to examine the role of genetic variants in a large number of candidate genes for premature, familial CAD and myocardial infarction (MI). Candidate genes were chosen for their acknowledged role in endothelial cell biology, vascular biology, lipid metabolism, and the coagulation cascade and their probable pathophysiologic link to thrombotic cardiovascular diseases. Statistical analysis showed and association of CAD and MI with the finding for SNPs in members of the thrombospondin gene family, particularly described herein, are SNPs in TSP-2.

Methods

Case Population

Fifteen medical centers in the United States (Appendix) participated in the enrollment of probands and their affected siblings. Each proband was required to have developed coronary heart disease by age 45, if male, or age 50, if female, as manifest by either a myocardial infarction, surgical or percutaneous coronary revascularization, or a coronary angiogram with evidence of at least a 70% stenosis in a major epicardial artery. At least one sibling who also has fulfilled these criteria had to be alive to qualify for inclusion, and the proband along with affected sibling(s) answered a health questionnaire, had anthropometric measures taken, and blood drawn for measurement of serum makers and extraction of DNA. The protocol was approved by the institutional review board at each participating institution. All patients gave informed consent to participate. For the purpose of this case-study, a series of unrelated singleton cases were selected such that only one affected individuals from each family was represented, giving preference to the sibling with the earlier age of onset. The case series was limited to Caucasian families as they represented the majority of the collection.

Control Subjects

Controls representing a general, unselected population were identified through random-digit phone dialing in the Atlanta, Ga. area. Subjects ranging in age from age 20 to age 70 were invited to participate in the study. The subjects were invited to the clinic where they answered a health questionnaire, had anthropometric measures taken, and blood drawn for measurement of serum makers and extraction of DNA. This protocol was approved by a regional institutional review board.

Variant Allele Discovery, Validation and Genotyping

Cell lines derived from an ethnically diverse population were obtained and used for single nucleotide polymorphism discovery by methods previously described in detail (Cargill, M. et al., “Characterization of single-nucleotide polymorphisms in coding regions of human genes,” Nature Genetics, 22:231-238 (1999)). Genomic sequencing representing the coding and partial regulatory regions of genes were amplified by polymerase chain reaction and screened via two independent methods: denaturing high performance liquid chromatography or variant detector arrays (Affymetrix). An average of 114 chromosomes were screened for each gene, providing 99% power to detect alleles of >5% frequency and 65% power to detect alleles of >1% frequency. Using these methods, the overall sensitivity of SNP discovery is in excess of 90% (Cargill, et al., ibid.). Sequencing was performed to validate each putative SNP, and genotyping was performed with single base extension with either fluorescence energy transfer or fluorescence polarization. At least one SNP from each of a total of 51 genes related to cardiovascular biology genes were assessed, for a total of 85 SNPs. SNPs were selected based on a preference for missense variation in protein sequences or high allele frequency in and around coding sequence. Seventeen variants were deemed to be too rare to justify genotyping in the complete set of cases and controls.

Statistical Analysis

All analysis were done using the SAS statistical package (Version 8.0, SAS Institute, Inc., Cary, N.C.). Differences between cases and controls were assessed with analysis of variance (ANOVA) for continuous covariates and a chi-square statistic for categorical covariates. Association between each SNP and two outcomes, CAD and MI, was measured by comparing genotype frequencies between controls and all CAD cases and the subset of cases with MI. Significance was determined using a continuity-adjusted chi-square or Fisher's exact test for each genotype compared to the homozygous wildtype for that locus. Odds ratios were calculated and presented with 95% confidence intervals.

Genotype groups were pooled for subsequent analyses of the top loci. Pooling allowed the testing of the best model for each locus (dominant, codominant or recessive). Models were chosen based on significant differences between genotypes within a locus. A recessive model was chosen when the homozygous variant differed significantly from both the heterozygous and homozygous wildtype, and the latter two did not differ from each other. A codominant model was chosen when homozygous variant genotypes differed from both heterozygous and homozygous wildtype, and the latter two differed significantly from each other. A dominant model was chosen when no significant difference was observed between heterozygous and homozygous variant genotypes.

Multivariate logistic regression was used to adjust for gender, age, presence of hypertension, diabetes and body mass index using the LOGISTIC procedure in SAS. Age was defined as age at diagnosis for cases and current age for controls. Height and weight, measured at the time of enrollment, was used to calculate body mass index for each subject. Presence of hypertension and non-insulin-dependent diabetes was measured by self-report (controls) and medical record confirmation (cases).

Significant differences in plasma levels of thrombospondin were assessed using the GENMOD procedure of SAS. This procedure took into account the repeated measures of thrombospondin on each sample (each was measured twice). Since the plasma levels of thrombospondin were not normally distributed, the data were log-transformed prior to analysis. Results were converted back to ng/mL for presentation of data. Results The demographic characteristics of the 352 cases and 418 controls are presented in Table 1. Cases and controls differed significantly for all covariates (p<0.0001). Cases were more likely than controls to be male, older, diabetic, hypertensive and have a higher body mass index. The most common event which led to inclusion of a case into the study was myocardial infarction (54%). Cases were enrolled in the study, on average, nine years following their qualifying event suggesting a survivor bias.

Genotype distributions for cases and controls are shown in Table 1 for all loci examined. Eleven SNPs in nine genes showed statistically significant differences between cases and controls for either CAD, MI or both (defined as p<0.05; Table 3). The genes included THBS1, THBS2, THBS4, HRG, PAI2, ANXA4, PLCG1 and MTHFR. All three of the associated SNPs within the PAM2 gene were in tight linkage disequilibrium with each other. A variant in only one of these genes, MTHFR (C677T), has been previously reported to be associated with CAD. This association was most pronounced in the patients suffering MI and limited to those individuals homozygous for the variant allele.

Table 2 shows the results of the analysis for TSP-2 (THBS2). For thrombospondin-4 (THBS4), the variant was a change from alanine (A) to proline (P) at condon 387 in the third type 2 repeat unit. The SNP for thrombospondin-1 (THBS1) involved a change from asparagine (N) to serine (S) at condon 700, which occurs in the first type 3 repeat unit of the thrombospondin-1 protein.

THBS2

For thrombospondin-2, a change in the 3′ untranslated region from a thymidine residue (t) to a guanine residue (g) was associated with a change in the incidence in coronary artery disease and myocardial infarction. Individuals homozygous for the variant allele (g) were protected from CAD (p=0.012). This association remained significant after adjusting for covariates and yielded an odds ratio of 0.43, p=0.017. When the MI cases were analyzed, the association became more pronounced and significant after adjusting for covariates (OR=0.27; p=0.011).

Given the interesting coincidental associations of variants in three thrombospondin family members with CAD or MI, we examined plasma levels of thrombospondin-1 using a commercially available ELISA assays. Patients who were homozygous for the variant (SS) had the highest odds ratio of MI (8.66).

TABLE 1
Demographic Characteristics

| | Cases | Controls |
|---|---|---|
| Characteristic | N = 352 | N = 418 |
| Gender (% male) | 246 (70%) | 182 (44%) |
| NIDDM (%) | 36 (10%) | 10 (2%) |
| Hypertension (%) | 154 (44%) | 53 (13%) |
| BMI (kg/m²); mean ± SD | 29.4 ± 5.7 | 26.8 ± 6.2 |
| (range) | (16-61) | (20-70) |
| Current age; mean ± SD | 48.1 ± 7.3 | 43.0 ± 14.3 |
| (range) | (29-74) | (20-70) |
| Age at Diagnosis | 39.3 ± 4.9 | N/A |
| (range) | (22-51) | |
| Qualifying Event: | | |
| Angiography | 54 (15%) | N/A |
| CABG | 53 (15%) | |
| MI | 190 (54%) | |
| PTCA | 42 (12%) | |
| Other | 13 (4%) | |

(All variable differed significantly (p < .0001) between cases and controls.)

TABLE 2

| | | | | | | | | | | |
|---|---|---|---|---|---|---|---|---|---|---|
| Gene | SNP | Flanking | Mutation | Genot | | CAD | MI | CAD | MI | |
| Name | ID | Sequence | Type | type | Controls | cases | cases | OR | OR | p < .05 |

| | | | | | | | | | | |
|---|---|---|---|---|---|---|---|---|---|---|
| THBS2 | G5755e5 | AATGGAAC[T/G]CAGAGATG | Non- | GG | 38 | 15 | 6 | 0.51 | 0.39 | 0 |
| | | | coding | | | | | (.27, .96) | (.16, .97) | |
| | | | | GT | 147 | 147 | 83 | 1.28 | 1.41 | |
| | | | | | | | | (.94, 1.75) | (.97, 2.04) | |
| | | | | TT | 199 | 155 | 80 | 1.00 | 1.00 | |
| THBS2 | G5755e | AAATGTAG[C/T]GACTGTCA | Non- | TT | 0 | 0 | 0 | NC | NC | |
| | | | coding | | | | | | | |
| | | | | TC | 6 | 4 | 3 | 0.84 | 1.17 | |
| | | | | | | | | (.24, 3.01) | (.29, 4.75) | |
| | | | | CC | 385 | 305 | 164 | 1.00 | 1.00 | |

While this invention has been particularly shown and described with references to preferred embodiments thereof, it will be understood by those skilled in the art that various changes in form and details may be made therein without departing from the scope of the invention encompassed by the appended claims.

1. A method of predicting the likelihood of a vascular disease in an individual, comprising determining the genotype of the individual at a nucleotide position of a thrombospondin-2 gene which corresponds to nucleotide position 3949 of SEQ ID NO: 1; wherein an individual who is homozygous for the variant allele at this nucleotide position has a decreased likelihood of a vascular disease as compared with an individual who is heterozygous or homozygous for the reference allele at this nucleotide position.

2. The method of claim 1, wherein the thrombospondin-2 gene has the nucleotide sequence of SEQ ID NO: 1.

3. The method of claim 1, wherein the vascular disease is selected from the group consisting of atherosclerosis, coronary heart disease, myocardial infarction, stroke, peripheral vascular diseases, venous thromboembolism and pulmonary embolism.

4. The method of claim 3, wherein the vascular disease is myocardial infarction.

5. The method of claim 3, wherein the vascular disease is coronary heart disease.

6. The method of claim 1, wherein the variant allele is a G.

7. The method of claim 1, wherein the reference allele is a T.

8. A method of predicting the likelihood of a vascular disease in an individual, comprising determining the genotype of the individual at a nucleotide position of a thrombospondin-2 gene which corresponds to nucleotide position 3949 of SEQ ID NO:1; wherein an individual who is heterozygous or homozygous for the reference allele at this nucleotide position has an increased likelihood of a vascular disease as compared with an individual who is homozygous for the variant allele at this nucleotide position.

9. The method according to claim 8, wherein the thrombospondin-2 gene has the nucleotide sequence of SEQ ID NO: 1.

10. The method according to claim 8, wherein the vascular disease is selected from the group consisting of atherosclerosis, coronary heart disease, myocardial infarction, stroke, peripheral vascular diseases, venous thromboembolism and pulmonary embolism.

11. The method according to claim 10, wherein the vascular disease is myocardial infarction.

12. The method according to claim 10, wherein the vascular disease is coronary heart disease.

13. The method of claim 8, wherein the variant allele is a G.

14. The method of claim 8, wherein the reference allele is a T.

15. A method of diagnosing or aiding in the diagnosis of a vascular disease in an individual, comprising determining the nucleotide present at a nucleotide position of a thrombospondin-2 gene which corresponds to nucleotide position 3949 of SEQ ID NO: 1; wherein presence of a T at this nucleotide position is indicative of an increased likelihood of a vascular disease in the individual, as compared with an individual who is homozygous for the variant allele G at this nucleotide position.

16. A method of claim 15, wherein the vascular disease is selected from the group consisting of atherosclerosis, coronary heart disease, myocardial infarction, stroke, peripheral vascular diseases, venoms thromboembolism and pulmonary embolism.

17. A method of diagnosing or aiding in the diagnosis of a vascular disease in an individual, comprising determining the genotype of the individual at a nucleotide position of a thrombospondin-2 gene which corresponds to nucleotide position 3949 of SEQ ID NO: 1; wherein an individual who is homozygous for the variant allele at this nucleotide position has a decreased likelihood of a vascular disease as compared with an individual who is heterozygous or homozygous for the reference allele at this nucleotide position.

18. A method of claim 17, wherein the vascular disease is selected from the group consisting of atherosclerosis, coronary heart disease, myocardial infarction, stroke, peripheral vascular diseases, venoms thromboembolism and pulmonary embolism.

19. The method of claim 17, wherein the variant allele is a G.

20. The method of claim 17, wherein the reference allele is a T.
